(19) United States
(12) Patent Application Publication
(10) Pub. No.: US 2024/0374560 A1
(43) Pub. Date: Nov. 14, 2024

(54) FIBER-ENHANCED LIPOSOME AND METHODS

(71) Applicant: Vidya Herbs, Inc., Red Bank, NJ (US)

(72) Inventor: Kodimule Shyam PRASAD, Bangalore (IN)

(73) Assignee: Vidya Herbs, Inc., Red Bank, NJ (US)

(21) Appl. No.: 18/662,189

(22) Filed: May 13, 2024

Related U.S. Application Data
(60) Provisional application No. 63/528,412, filed on Jul. 23, 2023.
(60) Provisional application No. 63/466,326, filed on May 14, 2023.

(51) Int. Cl. A61K 31/375; A61K 9/127; A61K 31/4375; A61K 31/522; A61K 31/56; A61K 38/39; A61K 47/24; A61K 47/36; A61K 47/46

(52) U.S. Cl. CPC A61K 31/375; A61K 9/1272; A61K 31/4375; A61K 31/522; A61K 31/56; A61K 38/39; A61K 47/24; A61K 47/36; A61K 47/46

(57) ABSTRACT

The invention provides a liposome with integrated dietary fiber. The inventive liposome has improved stability, strength, encapsulation and delivery properties for hydrophobic and hydrophilic agents. The integrated dietary fiber permits the liposome to be dried for enhanced storage stability without compromising liposome integrity, strength, shape or surface smoothness due to the loss of water. Methods of making and using the inventive liposome for the delivery of active agents are also contemplated by the invention.

### CROSS-REFERENCE TO RELATED APPLICATIONS

This application claims the benefit of provisional application No. 63/528,412, filed Jul. 23, 2023, and provisional application No. 63/466,326, filed May 14, 2023. The entire contents of these applications are incorporated herein by reference for all purposes.

### BACKGROUND

Liposomes are small spherical structures composed of a phospholipid bilayer that resembles a cell membrane. Liposomes provide delivery vehicles for transporting active molecules into the body by facilitating absorption through the gut membrane by preventing breakdown of the active molecules by stomach acid. Liposome-encapsulated active molecules may mimic the body's own transport system, enhancing uptake and delivery. Phospholipids are amphiphilic molecules, containing polar and nonpolar portions, and provide an excellent encapsulation medium.

Liposomes can encapsulate and transport both hydrophilic agents and hydrophobic agents through the gut membrane (GI tract) thereby improving their permeability. Moreover, biodegradable lipid molecules can improve transcellular transport via transient disruption of cellular lipophilic bilayers and may well enhance paracellular drug transport. Liposomal encapsulation of active agents improves their stability and bioavailability and enhances their controlled release within the GI tract. Thus, liposome delivery systems offer a promising solution for the delivery of drugs and other less bioavailable active agents. Liposomes can also provide active agents with improved bioavailability, targeted delivery, increased circulation time, enhanced solubility, reduced toxicity, and greater stability.

Despite their advantages, conventional liposomes have many limitations. For example, the phospholipids of conventional liposome formulations can undergo aggregation, flocculation, fusion and coalescence which changes the size and integrity of the liposomes and resulting in the seepage of encapsulated agents. Thus, agents encapsulated in conventional liposomes can have a relatively fast rate of clearance from the body as they are permitted to seep out of the liposome delivery vehicle. The aggregation, flocculation, fusion and coalescence of conventional liposome formulations can also lead to a modification of encapsulated agents. Additionally, the physical and chemical instability of conventional liposome formulations can exacerbate these effects when the liposomes are stored over time. Thus, conventional liposome formulations can fail to protect, and even impair, the therapeutic effect of the agents they are used to encapsulate.

Another disadvantage of conventional liposomes is that they are typically provided in a hydrated form wherein water is contained within the core of the liposomes. For example, liposomes and their encapsulated agents are often suspended in liquids and pastes. In their hydrated form, liposomes are relatively weak and subject to breakage. Moreover, storing liposomes over time in a hydrated form can lead to the degradation of encapsulated agents that are susceptible to aqueous environments. While drying liposomes may be attempted to avoid these problems, drying conventional liposome formulations leads to their collapse as the phospholipids are incapable of maintaining liposome integrity as water is removed from the core of the liposomes.

What is needed in the art therefore is an improved liposome having an enhanced physical and chemical stability that avoids the aggregation, flocculation, fusion and coalescence of phospholipids that occurs in conventional liposome formulations. The art also needs a liposome that can be dried while avoiding the breaking of liposomes that typically occurs in conventional liposomes due to the loss of water from the liposome core.

### BRIEF DESCRIPTION OF THE DRAWINGS

FIG. 1 shows a schematic diagram for a process of making an embodiment of the inventive liposome.

FIG. 2. shows the comparative release profile of vitamin C encapsulated in liposomes of the invention versus control samples. The values are mean±SD of three independent experiments. Data were analyzed by paired t test. *p<0.01 and **p<0.001 versus control.

FIG. 3 shows the release rate of vitamin C during simulated gastrointestinal digestion. The inventive liposomal formulation sample was incubated separately in simulated gastric fluid (SGF) and simulated intestinal fluid (SIF) at 37° C. for 4 h. The release of vitamin C was quantified at different time points. The values are mean±SD of three independent experiments. The data were analyzed by paired t test. *p<0.05 and **p<0.01 versus SGF.

FIG. 4 shows the comparative release profile of berberine formulated with the inventive liposome versus control, non-encapsulated berberine. The values are mean±SD of three independent experiments.

FIG. 5 shows the 24-h kinetics of berberine plasma concentrations after a single oral administration of berberine formulated with the inventive liposome versus control berberine. The data are presented as mean±standard deviation (SD) of three independent experiments.

FIG. 6 shows the graphical representation of mean plasma concentration of vitamin C after oral administration of a single dose of vitamin C formulated with the inventive liposome versus non-encapsulated vitamin C.

FIG. 7 shows the 24-h kinetics of plasma concentrations of ursolic acid after a single oral administration of ursolic acid encapsulated with the inventive liposome versus non-encapsulated ursolic acid (control). The data are presented as mean±standard deviation (SD) of three independent experiments.

FIG. 8 shows the comparative release profile of caffeine formulated with the inventive liposome versus non-encapsulated caffeine (control). The values are mean±SD of three independent experiments. The data were analyzed by paired t test. **p<0.01 and ***p<0.001 Vs. control.

FIG. 9 shows the comparative release profile of glutathione formulated with the inventive liposome versus non-encapsulated glutathione (control). The values are mean±SD of three independent experiments.

FIG. 10 shows the release rate of a glutathione formulated with the inventive liposome during simulated gastrointestinal digestion. The values are mean±SD of three independent experiments.

FIG. 11 shows the plasma concentration-time curve for total hydroxyproline (Hyp) after oral administration of collagen encapsulated in the inventive liposome in rats. The values are mean±SD (n=3 of pooled plasma samples). The data were analyzed by two-way ANOVA followed by Šídák's multiple comparison test. ***p<0.0001, **p<0.001 and *p<0.01 between the treatments.

### DETAILED DESCRIPTION

The invention provides an improved liposome formulation that integrates fiber into the liposome structure. Without being limited to any particular theory or mechanism, combining fiber with the liposome's phospholipid bilayer enhances the liposome's physical and chemical stability. Thus, the inventive liposome avoids the aggregation, flocculation, fusion and coalescence problems that characterize conventional liposomes.

Without being limited to any particular theory or mechanism, integrating fiber into the liposome can prevent or inhibit the collapse of the liposome structure during drying which typically destroys the structure of conventional liposomes. Thus, the inventive liposome can be used to provide stabilized powdered liposomal formulations. This is particularly advantageous because powdered liposomal formulations can be stored for an extended period of time without compromising the structure, integrity and stability of the liposomes so that their encapsulation and delivery properties can be maintained. Moreover, powdered liposomal formulations protect encapsulated agents from aqueous degradation and leaking. Powdered liposomal formulations also protect encapsulated agents from aggregation with the phospholipids that form the liposome shell.

Without being bound by any particular theory or mechanism, the integrated fiber of the inventive liposome provides a high surface area that traps water and oils due to the amphiphilic nature of the fiber. Without being bound by any particular theory or mechanism, the lipophilic and hydrophilic ends of the fiber can bind with lecithin to reinforce the liposome structure thereby increasing the liposome's strength and ability to cross the gut wall due to fiber's amphiphilic nature. Thus, the high surface area and amphiphilic properties of fiber, can permit the fiber to be used at very low levels while providing high functional benefits such as high water holding, surface area, encapsulation, emulsification and gelling properties. The fiber can also function as a secondary dietary aid and prebiotic.

Without wishing to be bound to any particular theory or mechanism, integrating fiber into the inventive liposome's bilayer can also produce liposomes that are smoother and more spherical in shape (i.e., less angular) than conventional liposomes. Thus, the smoother, more spherical shape of the inventive liposome decreases its surface contact angles to make it more dispersible in water.

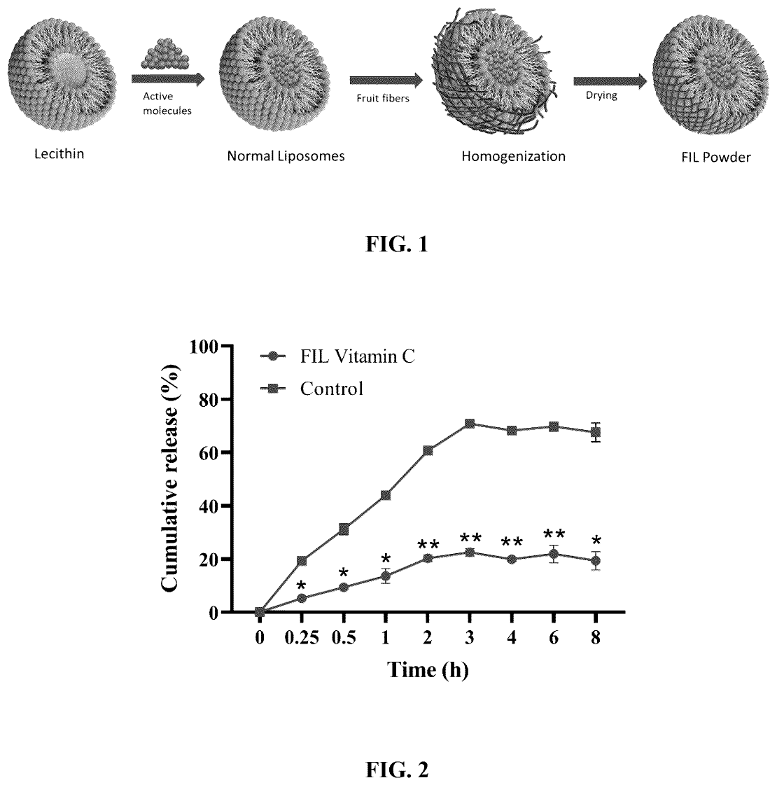

FIG. 1 shows a schematic representation of a non-limiting method of making the inventive liposome. The method can be practiced by providing a liposome formed of phospholipids (lecithin in this example) and combining the liposome with one or more active agents under conditions suitable for producing a liposome that encapsulates the one or more active agents. The liposome encapsulating the one or more active agents is then combined with fiber (e.g., dietary fiber) and agitated (e.g., homogenized) under conditions sufficient to integrate the fiber into the liposome. The liposome having fiber integrated therein is then dried to produce a powdered, fiber-enhanced liposome that encapsulates the one or more active agents.

The method of making the inventive liposome can also be practiced by combining one or more phospholipids, one or more types of fiber, and one or more active agents in a liquid medium. In some aspects, the liposomes are practiced by combining one or more phospholipids, one or more types of fiber, starch (e.g., modified food starch) and one or more active agents in a liquid medium. The combined medium is then agitated under conditions suitable for producing liposomes that encapsulate the one or more active agents, wherein the liposomes have the one or more types of fiber integrated within the liposomes' structure. The liposomes are then dried to produce a powdered formulation of fiber-enhanced liposomes that encapsulate the one or more active agents. The inventive liposome can comprise 10-40% (w/w) phospholipids and 3-15% (w/w) fiber. In other aspects, the liposomes can comprise 10-40% (w/w) phospholipids, 3-15% (w/w) fiber and 10-60% (w/w/) starch. Without being bound by any particular theory or mechanism, the incorporation of starch into the inventive liposome formulation provides a coating that can increase the thermal stability of the liposome.

The inventive liposome can be made by homogenization, mixing (e.g., micromixing) sonication, detergent dialysis, ethanol injection, extrusion (e.g., high pressure extrusion and French press extrusion), ether infusion, the Mozafari method, or reverse phase evaporation. Alternatively, the inventive liposomes can be made by agitating lipids in the presence of water to form multilamellar vesicles which can then be subjected to several cycles of freeze-thawing in order to increase the trapping efficiencies for water soluble drugs. Alternatively, the inventive liposomes can be made by using multilamellar vesicles as a starting material. The liposomes of the invention can be multilamellar vesicles, unilamellar vesicles, cochleate vesicles, or combinations thereof. The liposomes of the invention can be multivesicular liposomes in which one vesicle contains one or more smaller vesicles.

While FIG. 1 depicts a method of making a liposomal composition of the invention using lecithin, it will be appreciated that the inventive liposome can incorporate any phospholipid or combination of phospholipids capable of encapsulating a desired agent (e.g., bioactive molecule or other substance). In preferred embodiments, the phospholipid or combination of phospholipids is suitable for delivery into the body of subject. The subject can be a mammal, including humans and animal subjects.

Some non-limiting examples of phospholipids for use with the invention include phosphatidylserine, phosphatidylcholine, phosphatidylethanolamine, sphingomyelin, phosphatidylinositol, phosphatidic acid, cholesterol, or combinations thereof. The phospholipids can be glycerophospholipids, phosphosphingolipids, or a combination thereof. Some non-limiting examples of phosphosphingolipids for use with the invention include ceramide phosphorylcholine, ceramide phosphorylethanolamine, ceramide phosphoryllipid, or combinations thereof.

Phospholipids for use with the invention can be natural phospholipids, synthetic phospholipids, or a combination thereof. Non-limiting examples of natural phospholipids for use with the invention include phospholipids obtained from one or more of egg yolk, milk and other animal-based materials, and phospholipids obtained from plant materials (e.g., one or more of soybeans, rapeseeds, sunflower seeds and cotton seeds). In some preferred embodiments, the liposomes are made with sunflower lecithin.

In some embodiments, the liposomes are integrated with one or more types of fiber. As used herein, the term “integrated,” and the like, refers to dietary fiber being interposed between the phospholipid molecules of the liposome's phospholipid bilayer, encapsulated within the liposome, on the inside surface of the liposome, on the outside surface of the liposome, or combinations thereof. While the inventive liposome is disclosed as being integrated with fiber, it will be appreciated that the invention can provide a composition comprising a mixture of liposomes and fiber, wherein at least a portion of the fiber is not integrated with the liposomes.

Fiber for use with the invention can be dietary fiber. The dietary fiber can be insoluble dietary fiber, soluble dietary fiber, or a combination thereof. Dietary fiber for use with the invention includes, but is not necessarily limited to, plant-derived dietary fiber. The plant-derived dietary fiber can be fruit fiber, vegetable fiber, or a combination thereof. Some non-limiting examples of plant-derived dietary fiber for use with the invention include oat fiber, pea fiber, bean fiber, apple fiber, citrus fruit fiber, carrot fiber, barley fiber, psyllium fiber, or combinations thereof. In some preferred embodiments, the fiber comprises citrus peel fiber, apple peel fiber, or a combination thereof. The use of dietary fiber provides the inventive liposome with the additional benefit of providing a subject with a source of fiber intake, while simultaneously delivering an encapsulated agent to the subject.

In preferred embodiments, the dietary fiber has a high surface area that easily traps water and oil due to the amphiphilic nature of the fiber. Without being bound to any particular theory or mechanism, the lipophilic and hydrophilic ends of the dietary fiber bind to the liposome phospholipids to reinforce the liposome thereby permitting the liposome to be dried while retaining its structure (i.e., preventing the collapse of the liposome as water is removed from the liposome core). In addition, the amphiphilic nature of the dietary fiber enhances the liposome's ability to cross the gut wall. Meanwhile, the high surface area of the dietary fiber permits relatively small amounts of dietary fiber to be used while providing water retention, a high surface area and encapsulation, emulsification and gelling properties. Thus, the inventive liposome can be effectively rehydrated if an aqueous delivery form is desired, such as after storage and/or distribution of a liposomal product as in commercial nutritional supplement applications, for example.

The liposomes of the invention can be used to encapsulate a wide variety of agents, or combination of agents, that are desired for delivery to a subject. The agent can be one or more active molecules. The agent can be a therapeutic, an agent for health maintenance, or a combination thereof. Agents for use with the invention can be natural or synthetic. Agents for use with the invention include, but are not necessarily limited to, vitamins, minerals, phytomolecules, terpenes (e.g., sesquiterpenes such as B-caryophyllene), biomolecules, (e.g., collagen) withanolides, steroids, small molecule drugs, and combinations thereof. Non-limiting examples of vitamins for use with the invention include vitamin C, vitamin B (e.g., vitamin B12), vitamin D, vitamin A, vitamin E, vitamin K, or combinations thereof. The phytomolecules can be one or more of flavonoids, polyphenols, phenolic acids (e.g., chlorogenic acids), isoflavones, curcumin, resveratrol, isothiocyanates, and carotenoids. The agent can be one or more botanical extracts. Suitable botanical extracts include, but are not necessarily limited to, green coffee bean extract, sunflower seed extract, turmeric rhizome extract, coffee fruit pulp extract, jackfruit extract, mango extract, ginger extract, saw palmetto extract, ashwagandha extract, Aframomum melegueta extract, or combinations thereof. Other suitable agents for use with the invention include, but are not necessarily limited to, ursolic acid, berberine, glutathione, caffeine, quercetin, CoQ10, lutein, melatonin, or combinations thereof. It will be understood that the liposomes of the invention can encapsulate one or a combination of the agents disclosed herein.

In one non-limiting embodiment, the liposome encapsulates a green coffee bean extract that includes a mixture of chlorogenic acids that consists of about 13% 3-caffeoylquinic acid (3-CQA), about 47% 5-caffeoylquinic acid (5-CQA), about 17% 4-caffeoylquinic acid (4-CQA), about 5% 5-feruloylquinic acid (5-FQA), about 6% 3,4-dicaffeoylquinic acid (3,4-diCQA), about 5% 3,5-dicaffeoylquinic acid (3,5-diCQA), and about 8% 4,5-dicaffeoylquinic acid (4,5-diCQA).

In one non-limiting embodiment, the liposome encapsulates a sunflower seed extract having a mixture of chlorogenic acids that consists of 4.1±1.42 w/w % 3-Caffeoylquinic acid (3-CQA), 28±4.65 w/w % 5-Caffeoylquinic acid (5-CQA), 6.5±2.25 w/w % 4-Caffeoylquinic acid (4-CQA), 0.84±0.26 w/w % 3,4-Dicaffeoylquinic acid (3,4-DICQA), 1.23±0.34 w/w % 3,5-Dicaffeoylquinic acid (3,5-DiCQA), and 1.85±0.42 w/w % 4,5-Dicaffeoylquinic acid (4,5-DiCQA).

In one non-limiting embodiment, the liposome encapsulates a turmeric rhizome extract having 75±5% w/w bisdemethoxycurcumin, about 10±5% w/w demethoxycurcumin and about 1.2±0.8% w/w curcumin.

The agent can be one or more hydrophilic agents, one or more lipophilic agents, or a combination thereof. Highly water-soluble agents like vitamin C are degraded quickly in the stomach by bile acids thus reducing their bioavailability. On the other hand, lipophilic active agents, like curcumin for example, may not get absorbed through the GI tract due to the agents' low water solubility. Without wishing to be bound to any particular theory or mechanism, integrating liposomes with soluble and insoluble fiber (e.g., dietary fiber) as disclosed herein can provide an equilibrium between hydrophilicity and lipophilicity that increases the bioavailability of both hydrophilic and lipophilic agents encapsulated in the inventive liposome.

Simultaneously, the encapsulation of hydrophilic agents, such as vitamin C for example, protects the agents from bile acid in the stomach thereby improving the agents' bioavailability and efficacy while providing a sustained release mechanism.

In some embodiments, the liposomes of the invention encapsulate vitamin C. Vitamin C is easily degraded in an aqueous medium, at high pH, and in the presence of oxygen and metal ions. Conventional vitamin C is typically provided in a crystalline formulation. While initially anhydrous, crystalline vitamin C is degraded by environmental moisture over time. In addition, crystalline vitamin C is largely degraded in the high pH environment of the stomach. Encapsulating vitamin C in the inventive liposome provides a significant improvement over conventional vitamin C formulations as it can protect vitamin C from environmental degradation due to moisture and oxidation. Thus, the inventive liposome permits vitamin C to be stored over without losing its bioactive properties. Additionally, encapsulating vitamin C using the inventive liposome prevents vitamin C from being prematurely degraded in the high pH environment of the stomach. This improves the bioavailability of vitamin C and provides a delayed release mechanism as active vitamin C is subsequently available for absorption in the small intestine. Integrating fiber in the inventive liposome as disclosed herein also prevents the phospholipids of the liposome's bilayer from interacting with, and altering, vitamin C. This effect provides an improvement over known liposome technologies by permitting encapsulated vitamin C to remain in its active form. Finally, integrating fiber in the inventive liposome permits the liposome to be dried without the loss of the liposome's structure which characterizes the drying of known liposomes which collapse when water is removed from the liposome core.

In some embodiments of the invention, the liposomes are free of water or substantially free of water. As used herein, the phrase “substantially free of water” means a water content that is less than about 20% w/w, less than about 10% w/w, less than about 5% w/w, less than about 4% w/w, less than about 3% w/w, less than about 2% w/w, or less than about 1% w/w water. As used herein, the term “about” means the stated quantity, value, amount or volume, or the stated quantity, value, amount or volume that varies (plus or minus) by up to 20%, up to 10%, or up to 5%. The liposome composition of the invention can be powdered.

In some embodiments, the liposomes of the invention are combined with one or more excipients. The liposomes can be combined with the one or more excipients by, for example, stirring, mixing, folding, or blending. The liposomes can be suspended in one or more excipients. In some embodiments, the liposomes and the one or more excipients are free of water or substantially free of water. The excipients can be selected based the properties of the desired dosage form for the liposomes and/or the excipients' compatibility with the liposomes and/or the agents encapsulated in the liposomes. Suitable excipients include, but are not necessarily limited to, binders, fillers, flow aids/glidants, disintegrants, lubricants, stabilizers, surfactants, and the like. The excipients can be one or more artificial excipients. Suitable excipients include, but are not necessarily limited to, those disclosed in the following references: Remington: The Science and Practice of Pharmacy, 19th Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N. Y.: Marcel Decker 1980); and Pharmaceutical Dosage Forms and Drug Delivery Systems, Seventh Ed (Lippincott Williams & Wilkins 1999). The entire contents of these and documents are incorporated herein by reference for all purposes.

In some embodiments, the invention provides a method of administering liposomes. The method can be practiced by providing a composition comprising the inventive liposomes having one or more agents encapsulated as disclosed herein, and administering the composition to a subject in need of the therapeutic or health maintenance effect of the one or more agents. The composition can be provided to a user in dry form to permit the user to hydrate the composition before administration. For example, the composition can comprise the inventive liposomes encapsulating one or more dietary or nutritional supplement agents wherein the composition is hydrated before oral administration. Alternatively, the composition can comprise the inventive liposomes encapsulating one or more agents for skin care wherein the composition is hydrated before topical administration.

The composition comprising the inventive liposomes can be administered systemically and/or locally. Suitable administration routes include auricular, buccal, conjunctival, cutaneous, dental, endocervical, endosinusal, endotracheal, enteral, epidural, extra-amniotic, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal dental, intracoronary, intracorporus cavernosum, intradermal, intradiscal, intraductal, intraduodenal, intradural, intraepidermal, intraesophageal, intragastric, intravaginal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parentera, percutaneous, periarticular, peridural, perineural, periodontal, rectal, inhalation, retrobulbar, soft tissue, subarachnoid, subconjunctival, subcutaneous, sublingual, submucosal, topical, transdermal, transmucosal, transplacental, transtracheal, transtympanic, ureteral, urethral, vaginal, or combinations thereof. The composition can be administered by irrigation, drip, infusion, or topically by a dressing, patch, or bandage that is in contact with the composition. In a preferred embodiment, the composition is administered orally. The subject can be a human or non-human animal, such as dogs, cats and other companion animals, livestock such as cattle, sheep, horses, pigs and goats, or laboratory animals such as mice, rats, guinea pigs and rabbits. The composition can be administered one or more times.

The composition can be administered to the subject in an administration form selected from a powder, liquid, pill, tablet, pellet, capsule, thin film, solution, spray, syrup, linctus, lozenge, pastille, chewing gum, paste, vapor, suspension, emulsion, ointment, cream, lotion, liniment, drop, topical patch, buccal patch, bead, gummy, gel, sol, injection, or combinations thereof. In some preferred embodiments, the composition is free of water, or substantially free of water, and is administered in form selected from a powder, pill, tablet, pellet, capsule, lozenge, or bead.

### EXAMPLE 1—LIPOSOME MANUFACTURE

20 g of sunflower lecithin was dissolved in 500 mL of Millipore hot water (80-85° C.) by continuous stirring. For the resultant mixture, 45 g to 70 g of Vitamin C was added and continuously sheared by high shear mixing to form true liposomes, also the temperature was lowered to 40° C. for 20 min. 10 g of fruit fiber (isolated from apple or citrus) was blended in under high shear for 10 min for the further encapsulation of the lecithin. To the homogenized mixture, 25 g of modified food starch was added during the high shear mixing. Then, the mixture was further homogenized at a pressure range of 300 to 500 bars. After that, the formulation was transferred through a spray dryer for drying, which had an intake temperature range of 160-170° C. and an exit temperature range of 60-70° C. The liposome composition obtained was put into vials and stored for later analysis at room temperature.

### EXAMPLE 2—SUSTAINED RELEASE STUDY OF ENCAPSULATED VITAMIN C

The comparative in vitro release study of vitamin C encapsulated in the inventive liposome versus and non-encapsulated vitamin C (control) was performed using the dialysis set up. Prior to the diffusion experiment, the cellulose acetate dialysis membrane (Sigma Aldrich, Molecular weight cut-off (MWCO) 12 kDa) was soaked in phosphate buffered saline (PBS, pH 7.4) for 12 h. A weighed quantity of encapsulated vitamin C (50 mg) was suspended in 10 mL of PBS and transferred into the dialysis bag (donor compartment). The dialysis bag was placed in a beaker containing 100 mL of PBS (receptor compartment). The setup was incubated at 120 rpm for 8 h in a 37° C. shaker incubator. A control experiment was performed simultaneously in which unencapsulated vitamin C solution was dialyzed. A 1 mL aliquot of PBS in the receptor compartment was sampled at different time points and an equal quantity of fresh PBS was replaced in the compartment after each withdrawal. The absorbance of the samples was measured at 295 nm. The concentration of vitamin C in the samples was determined using a standard curve.

### In Vitro Simulated Digestion Analysis

The encapsulated vitamin C was investigated for in vitro release and digestive stability using simulated gastric fluid (SGF) and intestinal fluid (SIF) models. The liposomes were added to the respective digestive juice at 2 mg/mL and incubated at 37° C. and 120 rpm. The samples were taken at regular intervals (0, 5, 15, 30, 60, 90 and 120 min) and centrifuged at 5000 rpm for 5 min. The absorbance was measured at 295 nm and the concentration of vitamin C determined.

### Results

FIG. 2 shows the in vitro release profiles of vitamin C from encapsulated vitamin C and control. There was an exponential increase in cumulative drug release up to 3 h in the case of the control whereas a sustained release pattern was seen in the encapsulated vitamin C formulation. The release of vitamin C was significantly less in the encapsulated vitamin C formulation as compared to the control at respective time intervals (p<0.01). After 8 h, the vitamin C released from the encapsulated vitamin C formulation and control samples were 19.31% and 67.54% respectively. These results suggest that the liposomal encapsulation inhibited the release of vitamin C which may be favorable for prolonged release applications.

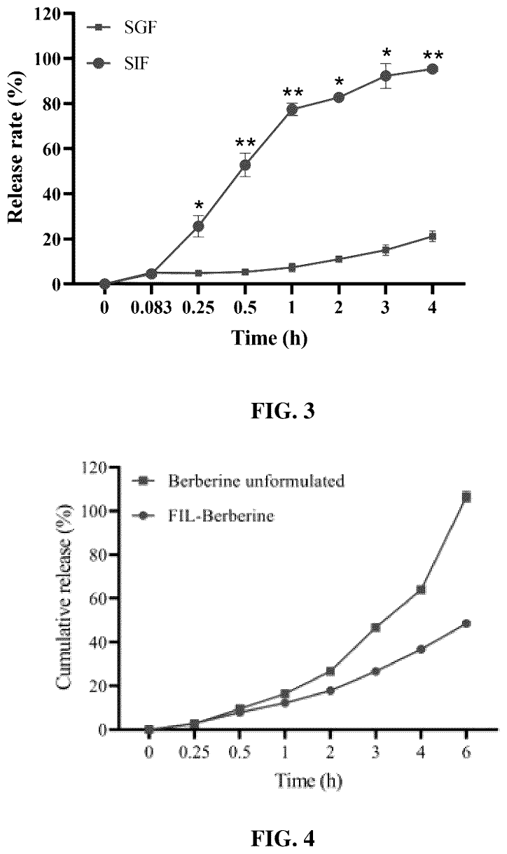

In vitro digestion of encapsulated vitamin C was performed with simulated gastric and intestinal fluids. As shown in FIG. 3, the release of vitamin C from the encapsulated vitamin C formulation in SGF was relatively slower compared to that in SIF. The release rate of vitamin C reached 21.15% after 4 h in SGF whereas in SIF the rate of vitamin C release was 95.34% at 4 h. This gives a clear indication of the better stability of agents in the stomach when encapsulated using the liposome of the invention. The results also show agents encapsulated in the liposomes of the invention have much better release and absorption in the GI tract. Usually, non-encapsulated vitamin C degrades easily in the stomach acids with the interaction of bile acids, but these results give the justification for the higher bioavailability of agents encapsulated with the inventive liposome.

### EXAMPLE 3—BIOAVAILABLE FORMULATION OF BERBERINE

### In Vitro Release Study

The present study was based on a bioavailable formulation of berberine (NLT 45% berberine) encapsulated in the inventive liposome. The present study demonstrated the in vitro release profile and the oral bioavailability of the encapsulated berberine relative to the non-encapsulated berberine (88% berberine HCl) (control).

The comparative in vitro release study of encapsulated berberine and non-encapsulated berberine was performed using the dialysis set up. Prior to the diffusion experiment, the cellulose acetate dialysis membrane (Sigma Aldrich, Molecular weight cut-off (MWCO) 12 kDa) was soaked in phosphate buffered saline (PBS, pH 7.4) for 12 h. A weighed quantity of encapsulated berberine (50 mg) was suspended in 10 mL of PBS and transferred into the dialysis bag (donor compartment). The dialysis bag was placed in a beaker containing 100 mL of PBS (receptor compartment). The setup was incubated at 120 rpm for 8 h in a 37° C. shaker incubator. A control experiment was performed simultaneously in which the unformulated berberine solution was dialyzed.

A 1 mL aliquot of PBS in the receptor compartment was sampled at different time points and an equal quantity of fresh PBS was replaced in the compartment after each withdrawal. The absorbance of the samples was measured at 420 nm using a plate reader (Tecan Infinite MPro). The concentration of berberine in the samples was determined using a standard calibration curve.

### Animals

Male Wistar rats (220-250 g) were procured from Biogen Laboratory Animal Facility, Bangalore, India. All animals were housed in air-conditioned rooms of the animal facility with controlled temperature (23±2° C.) and relative humidity (35-50%); under 12 h light/dark cycle. The animals had access to commercial rodent diet and water ad libitum. The study protocol was approved by the Institutional Animal Ethics Committee (IAEC) of Vidya Herbs Pvt Ltd, Bangalore, India (Approval No. VHPL/PCL/IAEC/01/2023). The animal experiments were conducted in accordance with the CPCSEA (The Committee for the Purpose of Control and Supervision of Experiments on Animals, Government of India) guidelines.

### Pharmacokinetic Study

The animals were acclimatized for 7 days and later randomized to two groups (n=6). Rats in each group were administered with a single oral dose of either encapsulated berberine (600 mg/kg) or equivalent dose of non-encapsulated berberine HCl (320 mg/kg) prepared as a suspension in distilled water. The animals were fasted overnight prior to the experiment, with free access to water.

Blood was withdrawn from retro-orbital plexus at 0 (before administration), 0.25, 0.5, 1, 2, 3, 4, 8, 12 and 24 h after administration of test samples. The blood samples collected in heparinized tubes were centrifuged at 3000 rpm for 10 min at 4° C. The plasma samples were stored at −80° C. for further analysis.

### Sample Preparation and Quantification of Berberine by LCMS Method

The samples were extracted using the method described elsewhere with slight modifications._{[7]} Briefly, 100 μL of plasma samples were mixed with 400 μL of methanol and centrifuged at 7000 rpm for 10 min to precipitate the proteins. The supernatant collected was injected into the LCMS/MS system. The calibration standards were prepared by spiking the berberine working standard (25, 50, 100, 250 and 500 ng/mL) into the blank rat plasma to prepare the final concentrations of 5, 10, 20, 50 and 100 ng/ml in 100 μL.

The quantification of berberine is done by using an Electrospray ionization source (ESI) in positive mode by multiple reaction monitored (MRM) method. The elution was performed by using 5 mM ammonium formate containing 0.1% Formic acid: acetonitrile (solvent A: solvent B) with a flow rate of 0.2 mL/min, column temperature 40° C. was maintained for isocratic elution (20:80) having 3.0 L/min Nebulizing gas flow (Nitrogen used as a nebulizing gas), 400° C. of heat block temperature, 10 L/min drying gas flow, 220° C. maintained for DL temperature, interference temperature was maintained for 300° C.

Pharmacokinetic analysis was performed using R statistical software (Version 4.5). The data were presented as mean±standard deviation (SD).

### Results

FIG. 4 shows the in vitro release profiles of berberine in the encapsulated formulation and non-encapsulated control, at pH 7.4. There was an exponential increase in the cumulative release up to 6 h in case of control berberine whereas a sustained release pattern was seen in the encapsulated formulation. The berberine release was considerably lesser in encapsulated formulation as compared to the unformulated control at respective time intervals. After 6 h, the release % from the encapsulated formulation was 48.52% whereas in the non-encapsulated berberine the cumulative release reached 100%. These results suggest that the liposomal encapsulation slow down the release of berberine which may be favorable for prolonged release applications.

FIG. 5 shows the concentration-time curve of berberine after oral administration in rats. The pharmacokinetic parameters are summarized in Table 1. The mean plasma concentration (Cₘₐₓ) of berberine in the encapsulated formulation reached a maximum value of 50.98±27.03 ng/ml and Tₘₐₓ of 0.25±0.00 h. On the contrary, the non-encapsulated berberine showed the Cₘₐₓ of 15.54±9.66 ng/ml and Tₘₐₓ of 0.25±0.00 h. Further, the encapsulated berberine administered group showed a higher t1/2 (7.90±1.58 h) than the non-encapsulated berberine group (7.32±0.61 h). Importantly, the area under the curve (AUC) of encapsulated berberine (1.38±0.55 ng·h·mL⁻¹) was markedly higher than the non-encapsulated berberine (0.41±0.04 ng·h·mL⁻¹). Based on these data, it can be concluded that the oral bioavailability of encapsulated berberine is 3.37-fold higher than that of the non-encapsulated berberine HCl in Wistar rats

TABLE 1
Pharmacokinetic Parameters of Berberine After a Single Oral Dose in Rats

| Test | | | | AUC₀₋ₜ | |
|---|---|---|---|---|---|
| formulation | Cₘₐₓ (ng/mL) | Tₘₐₓ (h) | t½ (h) | (ng · h/mL) | Fr |
| FIL-Berberine | 50.98 ± 27.03 | 0.25 ± 0.00 | 7.90 ± 1.58 | 1.38 ± 0.55 | 3.37 ± 1.24 |
| Control | 15.54 ± 9.66 | 0.25 ± 0.00 | 7.32 ± 0.61 | 0.41 ± 0.04 | |
| (Unformulated) | | | | | |

The values are presented as mean ± SD (n = 4 per group).

The present study demonstrates the sustained release pattern of berberine in encapsulated formulation compared to that in the non-encapsulated control. Further, it was confirmed that the relative oral bioavailability of berberine is markedly improved in the encapsulated formulation compared to non-encapsulated berberine. These experimental findings encourage the use of encapsulated berberine with enhanced absorption profile in functional ingredients.

### EXAMPLE 4—IN VIVO STUDY OF ORALLY DELIVERED OF VITAMIN C METHODOLOGY

The study was a randomized, single-dose, double-blind, active-control comparative, two-way crossover, 2-sequence study to assess the comparative oral bioavailability of vitamin C encapsulated with the inventive liposome (FIL-Vitamin C) versus non-encapsulated Vitamin C supplementation in healthy volunteers. The inventive liposome comprised at least 40% w/w Vitamin C.

A group of 12 healthy volunteers were screened on Visit 1 to confirm their eligibility. Subjects fulfilling the eligibility criteria were randomized into 1 of 2-treatment sequences on Visit 2. There was a washout period of 7 days each between the 2 dosing days. All subjects were housed in the R & D clinical study facility at least 2 hours before administration of the test and control agents and until completion of the 12-hour post dose assessment in each study period. Subjects returned to the study center for the second period. On the morning of Day 1, in each period, subjects in a fasting state (at least 10 hours before dosing) received one 600 mg capsule orally of their assigned formulation (Table 2) as per randomization scheme, with 200±2 mL of water at room temperature. The study duration was approximately 12 days (2 dosing days and 1 washout period of 7±1 days). A total of 10 blood samples were collected in each of the two study periods. The pre-dose samples (2 mL) were collected within 1 hour before dosing. The post-dose samples (1×2 mL) were collected at 30 min, 60 min, 2, 3, 4, 5, 6, 8 and 12 hours after dosing in vacuum tubes containing K2 EDTA. All blood samples were collected from an ante-cubital vein or any suitable forearm vein using an indwelling cannula. Before each blood sample is drawn, 1 mL of blood was discarded so as to prevent the saline diluted blood and heparin from interfering with the analysis.

TABLE 2
Dosage for Clinical Analysis

| | TREATMENT GROUP A | TREATMENT GROUP B |
|---|---|---|
| Product | FIL-Vitamin C | Unformulated Vitamin C |
| Dosage Form | Capsules | Capsules |
| Composition | Liposomal formulation | Unformulated Vitamin C |
| | (not less than 40% w/w | (not less than 98% w/w |
| | of vitamin C) | of vitamin C) |
| Route | Oral | Oral |
| Dose | 600 mg capsule containing | 600 mg capsule containing |
| | 500 mg of FIL-Vitamin C | 200 mg of unformulated |
| | and 100 mg Maltodextrin | Vitamin C and |
| | | 400 mg Maltodextrin |
| Dosing | One capsule of 600 mg | One capsule of 600 mg |
| Regimen | | |
| Treatment | Single dose | Single dose |
| duration | | |

### Pharmacokinetic Analysis

Plasma concentrations were measured using ultra performance liquid chromatographyelectrospray ionization-tandem mass spectrometry (UPLC-ESI-MS/MS), gas chromatography mass spectrometry (GC-MS/MS) and High-Performance Liquid Chromatography (HPLC). All the pharmacokinetic parameters were compared between the two treatment groups using Pair-t-test with 5% level of significance. All statistical analysis was performed using R statistical software version 4.2.1.

TABLE 3
Plasma Concentrations after Single Dose of Test Agent Versus Control

| | | Treatment A | | | Treatment B | | | |
|---|---|---|---|---|---|---|---|---|
| Time | | (FIL-Vitamin C) | | | (unformulated Vitamin C) | | | |
| points | | (N = 12) | | | (N = 12) | | | |
| (hrs) | | Mean | SD | SE | Mean | SD | SE | P-value # |
| 0 | | 0.00 | 0.00 | 0.00 | 0.00 | 0.00 | 0.00 | NA |
| 0.5 | hour | 1017.53 | 763.76 | 220.48 | 219.99 | 138.01 | 39.84 | 0.0041* |
| 1 | hour | 1431.96 | 987.08 | 284.94 | 502.39 | 252.83 | 72.99 | 0.0079* |
| 2 | hour | 1854.51 | 1167.55 | 337.04 | 728.44 | 551.76 | 159.28 | 0.0083* |
| 3 | hour | 2885.32 | 1293.48 | 373.40 | 680.46 | 368.52 | 106.38 | 0.0001** |
| 4 | hour | 4604.58 | 3734.96 | 1078.19 | 456.28 | 252.59 | 72.92 | 0.0027* |
| 5 | hour | 2554.69 | 1467.39 | 423.60 | 405.53 | 249.68 | 72.08 | 0.0003* |
| 6 | hour | 1957.19 | 1437.22 | 414.89 | 338.54 | 197.91 | 57.13 | 0.0025* |
| 8 | hour | 1801.21 | 1308.14 | 377.63 | 219.14 | 175.48 | 50.66 | 0.0015* |
| 12 | hour | 1214.48 | 1044.94 | 301.65 | 162.08 | 150.54 | 43.46 | 0.0051* |

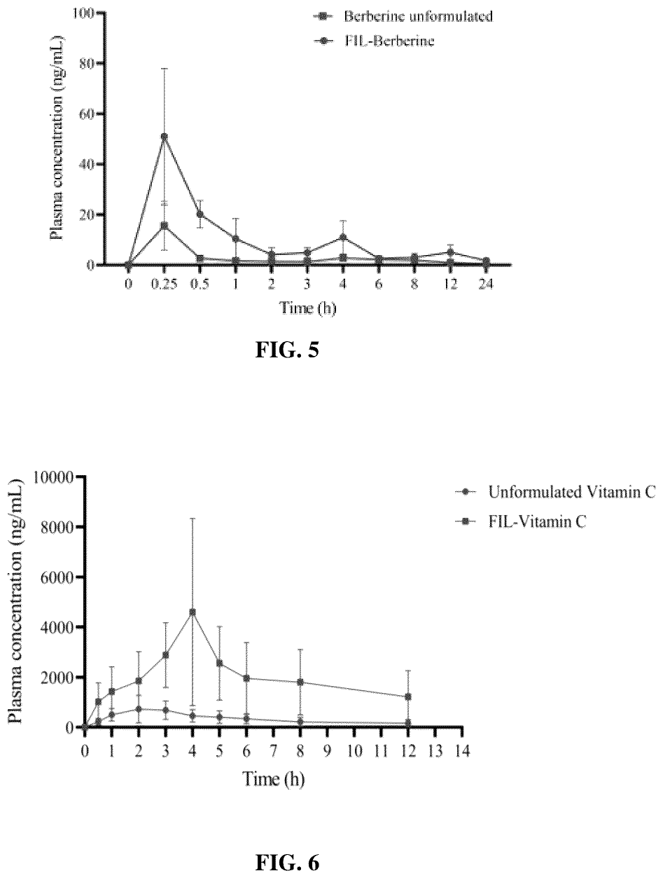

The plasma concentration of Vitamin C was found to be statically significant (p<0.05) higher at all the timepoints with highest levels (p<0.001) recorded at 3 hour timepoint following oral administration of single dose of encapsulated vitamin C compared to plasma concentration of vitamin C following oral administration of single dose of comparator, non-encapsulated vitamin C (FIG. 6 and Table 3).

TABLE 4
Pharmacokinetic Parameters after Single Dose of Test Agent Versus Control

| | Treatment A | | Treatment B | | |
|---|---|---|---|---|---|
| | (FIL-Vitamin C) | | (unformulated | | |
| | (N = 12) | | Vitamin C) (N = 12) | | ~P-value |
| Parameters | Mean | SD | Mean | SD | (A Vs B) |
| AUCₗₐₛₜ (ng · hr/mL) | 23802.96 | 13541.49 | 4126.30 | 1739.23 | 0.0004** |
| AUMCₗₐₛₜ (ng · hr²/mL) | 134257.00 | 85650.03 | 19660.17 | 10451.64 | 0.0007** |
| AUC_{inf} (ng · hr/mL) | 54661.75 | 72712.22 | 6258.67 | 4557.70 | 0.0418* |
| AUMC_{inf} (ng · hr²/mL) | 1815201.32 | 4527230.90 | 19660.17 | 10451.64 | 0.2144 |
| Cₘₐₓ(ng/ml) | 5111.46 | 3404.51 | 969.86 | 501.81 | 0.0014** |
| Tₘₐₓ (hour) | 3.75 | 0.87 | 2.67 | 1.07 | 0.0128* |
| MRT (hour) | 15.83 | 15.01 | 9.30 | 8.42 | 0.2062 |
| Kel (hour⁻¹) | 0.10 | 0.05 | 0.16 | 0.07 | 0.0277* |
| T_{half} (hour) | 10.38 | 10.61 | 5.75 | 6.09 | 0.2065 |

The AUCₗₐₛₜ [area under the concentration-time curve from dosing to the time of the last measured concentration≥lower limit of quantification (LLOQ)] for vitamin C in encapsulated vitamin C [23802.96±13541.49 (ng·hr/mL)] was found to be significantly (p<0.001) higher compared to non-encapsulated vitamin C [4126.30±1739.23 (ng·hr/mL)].

AUMCₗₐₛₜ [area under the moment curve from the time of dosing to the last measurable (positive) concentration] for vitamin C in encapsulated vitamin C [134257.00±85650.03 (ng·hr²/mL)] was found to be significantly (p<0.001) higher compared to non-encapsulated vitamin C [19660.17±10451.64 (ng·hr²/mL)].

AUC_{inf} indicates area under the plasma concentration-time curve extrapolated to infinity. The AUC_{inf} for vitamin C in encapsulated vitamin C [54661.75±72712.22 (ng·hr/mL)] was found to be significantly (p<0.001) higher compared to non-encapsulated vitamin C [6258.67±4557.70 (ng·hr/mL)] after the oral administration of a single dose.

AUMC_{inf} indicates the area under the moment curve from the time of dosing extrapolated to infinity. The AUMC_{inf} for vitamin C in encapsulated vitamin C [1815201.92±4527230.90 (ng·hr2/mL)] was found to be higher though not significantly (p=0.2144) compared to non-encapsulated vitamin C [92605.52±146390.90 (ng·hr₂/mL)] after the oral administration of a single dose.

The Cₘₐₓ [Maximum measured plasma concentration] for vitamin C in encapsulated vitamin C [5111.46±3404.51 (ng/ml)] was found to be significantly (p<0.001) higher compared to vitamin C in the encapsulated vitamin C group [969.86±501.81 (ng/ml)] after the oral administration of a single dose of encapsulated vitamin C and non-encapsulated vitamin C, respectively.

The Tₘₐₓ [time to the maximum measured plasma concentration] for vitamin C in FIL-Vitamin C [3.75±0.87 (hour)] was found to be significantly (p<0.05) higher compared to unformulated Vitamin C [2.67±1.07 (hour)] after the oral administration of a single dose.

The MRT [mean residence time] for vitamin C in encapsulated vitamin C [15.83±15.01 (hour)] was found to be higher though not significantly (p=0.2062) compared to non-encapsulated vitamin C [9.30±8.42 (hour)] after the oral administration of a single dose.

The Kₑₗ [elimination rate constant] for vitamin C in encapsulated vitamin C [0.10±0.05 (hour-1)] was found to be significantly (p<0.05) lower compared to non-encapsulated vitamin C [0.16±0.07 (hour-1)] after the oral administration of a single dose.

T_{half} [time to the maximum measured plasma concentration] for vitamin C in encapsulated vitamin C [10.38±10.61 (hour)] was found to be higher though not significantly (p=0.2065) compared to vitamin C in non-encapsulated vitamin C [5.75±6.09 (hour)] after the oral administration of a single dose.

Based on the findings of the study, the bioavailability of vitamin C following oral administration of single dose of test product (encapsulated vitamin C) was found to be higher compared to bioavailability of non-encapsulated vitamin C.

### EXAMPLE 5—BIOAVAILABILITY OF ENCAPSULATED URSOLIC ACID

Ursolic acid (UA) is a commonly adopted essential nutrient and bioactive compound which is added to various dietary supplements due to its health benefits. It can be found in a variety of plants, edible fruits, and herbs (e.g., apples, basil and holy basil, bilberries, cranberries, elderflower, peppermint, rosemary, lavender, oregano, thyme, hawthorn, and prunes). UA has been reported to have anti-inflammatory and hepatoprotective properties. UA can be used as promising ingredient to reduce obesity by increasing energy expenditure and the use of body fat.

Although UA presents the advantage of low toxicity, the clinical application of UA is limited due to low oral bioavailability due to its poor aqueous solubility, poor permeability, and metabolism by cytochrome P450 (CYP) isozymes, such as CYP3A4, all of which restrict UA's effectiveness. Another limitation is UA's nonspecific distribution throughout the body when administered intravenously. Thus, it is desirable to explore novel formulations of UA that overcome these problems. To overcome the UA's poor oral bioavailability and therapeutic issues, various strategies such as microencapsulation, using liposomes and nanoparticles, have been attempted. However, known encapsulation technologies have the disadvantages disclosed herein. Thus, the present study was conducted to evaluate the inventive liposome's use for encapsulating UA.

### Animals

Male Wistar rats (180-200 g) were procured from Biogen Laboratory Animal Facility, Bangalore, India. All animals were housed in air-conditioned rooms of the animal facility with controlled temperature (23±2° C.) and relative humidity (35-50%) under a 12 h light/dark cycle. The animals had access to a commercial rodent diet and water ad libitum. The animal study protocol was approved by the Institutional Animal Ethics Committee (IAEC) of Vidya Herbs Pvt Ltd, Bangalore, India (Approval No. VHPL/PCL/IAEC/08/2022). The animal experiments were conducted in accordance with the CPCSEA (The Committee for the Purpose of Control and Supervision of Experiments on Animals, Government of India) guidelines.

### Pharmacokinetics

The animals were acclimatized for 7 days and later randomized into two groups (n=6). Rats in each group were administered with a single oral dose of either encapsulated UA (300 mg/kg) or equivalent dose of non-encapsulated ursolic acid (60 mg/kg) prepared in a mixture of cremaphor, Tween 80, PEG, ethanol, and water (2:1:1:1:1.5). The animals were fasted overnight prior to the experiment, with free access to water. Blood was withdrawn from retro-orbital plexus at 0 (before administration), 0.25, 0.5, 1, 2, 4, 8, 12 and 24 h after administration of test samples. The blood samples collected in heparinized tubes were centrifuged at 3000 rpm for 10 min at 4° C. The plasma samples were stored at −80° C. for further analysis.

### Sample Preparation and Quantification of Ursolic Acid by LCMS Method

The samples were extracted using the method described elsewhere with slight modifications (Prasain et al. 2007). Briefly, 300 μL of pooled plasma samples spiked with internal standard (IS) betulinic acid (5 μg/mL) were acidified using 8 μL of glacial acetic acid. To this, 300 μL of methanol was added and centrifuged at 7000 rpm for 10 min to precipitate the proteins. The supernatant collected was injected into the LCMS/MS system. The calibration standards were prepared by spiking the ursolic acid working standard (25, 50, 100, 200, 400 and 800 ng/mL) and IS into blank rat plasma (200 μL).

The plasma concentration of UA was quantified using LCMS/MS 8050 System (Shimadzu, Japan) equipped with atmospheric pressure chemical ionization (APCI) source operating in positive ionization mode. The analysis was conducted on a Kinetex C18 column (100 Å, 150 mm×2.1 mm, 2.6 μm) at a flow rate of 0.2 mL/min. The mobile phase consisted of water:acetonitrile (A:B)=20:80 with isocratic elution and the injection volume of 5 μl.

Pharmacokinetic analysis was performed using R software. The data were analyzed by Mann Whitney U test using GraphPad Prism Version 9.5 (GraphPad Software Inc.). The data were presented as mean±standard deviation (SD) and considered statistically significant at p<0.05.

### Results

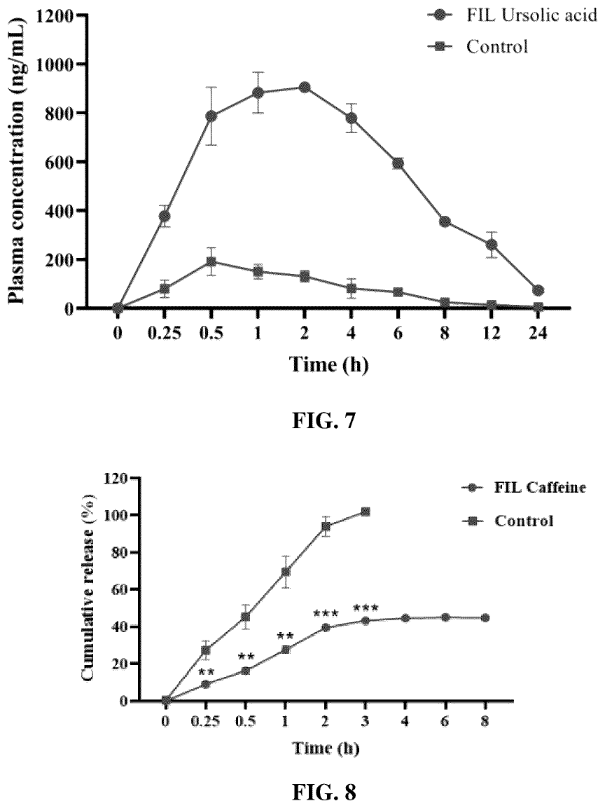

FIG. 7 shows the concentration-time curve of UA after oral administration in rats. The pharmacokinetic parameters are summarized in Table 5. The mean plasma concentration of UA from the encapsulated formulation reached maximum value of 930±16.97 ng/ml and Tmax of 1.5±0.71 h. On the contrary, the control group showed the maximum plasma concentration of 201±42.43 ng/ml and Tmax of 0.75±0.35 h for UA. Further, the encapsulated UA administered group showed a significantly higher t1/2 (6.60±0.24 h, p<0.01) and lower Kel (elimination constant) (0.11±0.007 h₋₁ p<0.001) compared to the control group (t1/2=4.49±0.11 h; Kel=0.15±0.004). Importantly, the area under the curve (AUC) of encapsulated UA (8464±196.4 ng·h₋₁·mL₋₁) was markedly higher than the control group (877.2±44.65 ng·h₋₁·mL₋₁). Based on these data, it can be concluded that encapsulated UA has a relative oral bioavailability 9.66 times more than the control.

TABLE 5
Pharmacokinetic Parameters of Encapsulated Ursolic Acid Versus Control Groups

| Test | Tₘₐₓ | Cₘₐₓ | t½ | Kel | AUC₀₋ₜₗₐₛₜ | |
|---|---|---|---|---|---|---|
| formulation | (h) | (μg/mL) | (h) | (h−1) | (ng · h−1 · mL−1) | Fr |
| FIL Ursolic acid | 1.5 ± 0.71** | 930 ± 16.97** | 6.60 ± 0.24** | 0.11 ± 0.007*** | 8464 ± 196.4*** | 9.66 ± 0.27 |
| Control | 0.75 ± 0.35 | 201 ± 42.43 | 4.49 ± 0.11 | 0.15 ± 0.004 | 877.2 ± 44.65 | |

The values are mean ± standard deviation (n = 3).
**p < 0.01 and
***p < 0.001 Vs. control.

### EXAMPLE 6—SUSTAINED RELEASE OF ENCAPSULATED CAFFEINE

The comparative in vitro release study of caffeine encapsulated in the inventive liposome versus plain caffeine (control) was performed using a dialysis set up. Prior to the diffusion experiment, the cellulose acetate dialysis membrane (Sigma Aldrich, Molecular weight cut-off (MWCO) 12 kDa) was soaked in phosphate buffered saline (PBS, pH 7.4) for 12 h. A weighed quantity of liposomal caffeine (50 mg) was suspended in 10 mL of PBS and transferred into the dialysis bag (donor compartment). The dialysis bag was placed in a beaker containing 100 mL of PBS (receptor compartment). The setup was incubated at 120 rpm for 8 h in a 37° C. shaker incubator.

A control experiment was performed simultaneously in which a plain caffeine solution was dialyzed. A 1 mL aliquot of PBS in the receptor compartment was sampled at different time points and an equal quantity of fresh PBS was replaced in the compartment after each withdrawal. The caffeine content in the samples were quantified using HPLC.

FIG. 8 shows the in vitro release profiles of caffeine from the encapsulated caffeine formulation versus control. There was an exponential increase in cumulative drug release up to 3 h in case of control whereas a sustained release pattern was seen in the encapsulated formulation. The release of caffeine was significantly lesser in the encapsulated formulation as compared to the control at respective time intervals (p<0.01). After 3 h, the caffeine released from the encapsulated formulation was 43.26% whereas in control the release of caffeine reached 100%. These results suggest that the liposomal encapsulation inhibited the release of caffeine which may be favorable for prolonged release applications.

### EXAMPLE 6—SUSTAINED RELEASE OF ENCAPSULATED CAFFEINE

Glutathione is an antioxidant present in cells. It plays a major role in detoxification of drugs. The hydrogen peroxide generated through cellular pathways is detoxified by reduced glutathione. As a dietary supplement, the safety of glutathione is well-established. It has several health benefits such as improving the liver function, diabetic complications, and skincare. It has been demonstrated through in vitro studies that glutathione has a skin-whitening effect and anti-aging properties.

The comparative in vitro release study of glutathione encapsulated in the inventive liposome and non-encapsulated glutathione (control) was performed using the dialysis set up. Prior to the diffusion experiment, the cellulose acetate dialysis membrane (Sigma Aldrich, Molecular weight cut-off (MWCO) 12 kDa) was soaked in phosphate buffered saline (PBS, pH 7.4) for 12 h. A weighed quantity of encapsulated glutathione (50 mg) was suspended in 10 mL of PBS and transferred into the dialysis bag (donor compartment). The dialysis bag was placed in a beaker containing 100 mL of PBS (receptor compartment). The setup was incubated at 120 rpm for 8 h in a 37° C. shaker incubator. A control experiment was performed simultaneously in which the non-encapsulated glutathione solution was dialyzed. A 1 mL aliquot of PBS in the receptor compartment was sampled at different time points and an equal quantity of fresh PBS was replaced in the compartment after each withdrawal. The concentration of glutathione in the samples was determined using the validated HPLC method.

The encapsulated glutathione was investigated for the digestive stability using simulated gastric fluid (SGF) and intestinal fluid (SIF) models. SGF was prepared by dissolving 200 mg of NaCl in 90 mL of distilled water and the pH of the solution adjusted to 1.2. Then 320 mg of pepsin was added, and the solution volume made up to 100 mL with distilled water. SIF was prepared by dissolving 680 mg of K2HPO4 into 80 mL of distilled water followed by adding 7.7 mL of 0.2 M NaOH. The pH of the solution was adjusted to 6.8. Then 1 g of trypsin was added, and the solution volume made up to 100 mL with distilled water.

The encapsulated glutathione was added to the respective digestive juice at 2 mg/mL and incubated at 37° C. and 120 rpm. The samples were taken at regular intervals (0, 5, 15, 30, 45, 60, 90, 120, 180, 240, 300 and 360 min) and centrifuged at 5000 rpm for 5 min. The supernatant was collected and analyzed for the glutathione content using HPLC.

The glutathione quantification was performed using RPHPLC PDA analytical method. The separation was done by isocratic elution at a flowrate of 0.9 mL/min in a Diol column 5 μm (250×4.6 mm). The mobile phase consisted of 50 mM phosphate buffer. The injection volume was 5 μL. The detection was carried out at 230 nm.

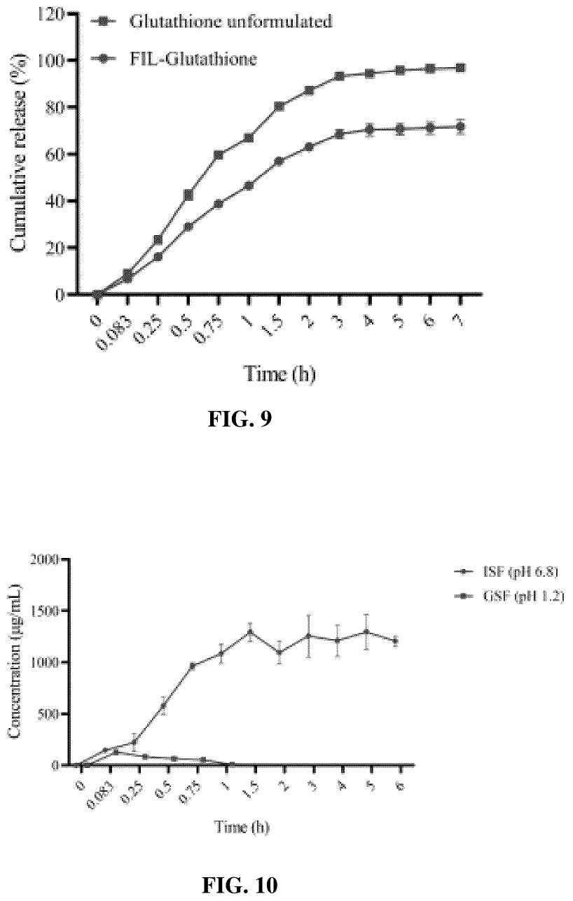

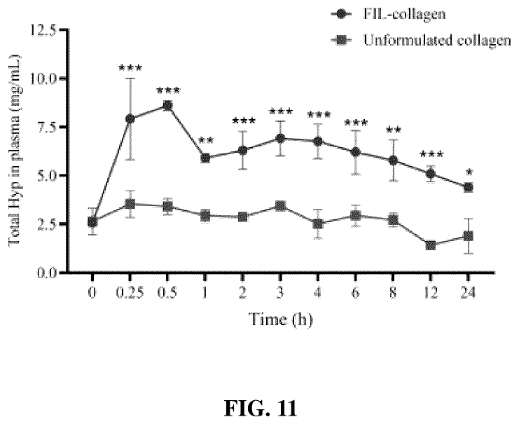

FIG. 9 shows the in vitro release profiles of glutathione in the encapsulated formulation versus the non-encapsulated control, at pH 7.4. There was an exponential increase in the cumulative release up to 4 h in case of control whereas a sustained release pattern was seen in the encapsulated formulation. The glutathione release was considerably less in the encapsulated formulation as compared to the non-encapsulated control at respective time intervals. After 7 h, the release % from the encapsulated formulation was 71.69% whereas in the non-encapsulated glutathione the cumulative release reached 96.86%. These results suggest that the inventive liposomal encapsulation formulation slows down the release of glutathione which may be favorable for prolonged release applications.

In vitro digestion of encapsulated glutathione was performed with simulated gastric and intestinal fluids. The encapsulated glutathione sample was incubated separately in simulated gastric fluid (SGF) and simulated intestinal fluid (SIF) at 37° C. for 4 h. The release of glutathione was quantified at different time points. As shown in FIG. 10, the release of glutathione from the encapsulated formulation in SGF was relatively lesser compared to that in the SIF. The release was considerably reduced in the gastric phase. After 1 h of incubation, there was no release observed. On the contrary, the release of glutathione in the intestinal phase increased exponentially up to 1.5 h and remained steady thereafter. There was a significant difference in the release pattern of glutathione in the gastric and intestinal simulated conditions. The results indicate that the liposomal encapsulation formulation protected the glutathione from releasing in the gastric phase thus showing the better stability of the encapsulated formulation in the gastrointestinal phase.

The present study demonstrates the sustained release pattern of glutathione in the inventive encapsulated formulation. Product stability in the encapsulated formulation was observed in the in vitro gastrointestinal phase. The use of glutathione formulated with the inventive liposome can find applications in cosmetics and dietary supplements to provide a sustained pattern release of desired agents thereby maintaining the optimal level of antioxidant availability for supporting cell physiology.

### EXAMPLE 6—BIOAVAILABILITY OF ORALLY ADMINISTERED ENCAPSULATED COLLAGEN

A large proportion of the total protein content of vertebrates is made up of collagen. Collagen hydrolysates have been used in traditional medicine for joint pain, to arrest bleeding and to improve blood circulation. Experimental evidence suggests the consumption of collagen provides beneficial effects against skin aging, osteoarthritis and osteoporosis, rheumatoid arthritis, and improvement in the health of nails and hair.

This study hypothesized that collagen supplement with liposomal encapsulation could slow the release of collagen in the intestine. Therefore, the objective of this study was to investigate collagen bioavailability in rats based on the pharmacokinetics of hydroxyproline (Hyp) in plasma after a single oral administration of collagen encapsulated with the inventive liposome.

Male Wistar rats (220-250 g) were procured from Adita Biosys Pvt Ltd., Bangalore, India. All animals were housed in air-conditioned rooms of the animal facility with controlled temperature (23±2° C.) and relative humidity (35-50%); under 12 h light/dark cycle. The animals had access to commercial rodent diet and water ad libitum. The study protocol was approved by the Institutional Animal Ethics Committee (IAEC) of Vidya Herbs Pvt Ltd, Bangalore, India. The animal experiments were conducted in accordance with the CCSEA (Committee for Control and Supervision of Experiments on Animals, Government of India) guidelines.

The animals were acclimatized for 7 days and later randomized to two groups (n=6). Rats in each group were administered with a single oral dose of either the encapsulated collagen formulation (total protein=46.14% total protein) (2000 mg/kg) or equivalent dose of unformulated collagen (total protein=90.75% protein) (1000 mg/kg) prepared as a suspension in distilled water. The animals were fasted overnight prior to the experiment with free access to water.

Blood was withdrawn from retro-orbital plexus at 0 (before administration), 0.25, 0.5, 1, 2, 3, 4, 6, 8, 12 and 24 h after administration of test samples. The blood samples collected in heparinized tubes were centrifuged at 3000 rpm for 10 min at 4° C. The plasma samples were stored at −80° C. for further analysis.

The bioavailability of collagen was indirectly evaluated by the bioavailability of Hyp in collagen, which was determined using a pharmacokinetic method after oral the administration. The Hyp concentration in plasma was determined by the colorimetric method.

Briefly, 100 μL of pooled plasma sample was mixed with 100 μL of 4N NaOH and autoclaved at 120° C. and 15 psi above atmospheric pressure for 15 min. The samples were cooled to room temperature and then neutralized by adding 100 μL of 4N HCl. Then, 625 L of chloramine-T solution was added to convert the Hyp to pyrolle-2-carboxylate by oxidation. The reaction mixture was allowed to stand at room temperature for 20 min followed by addition of 625 μL of Ehrlich's solution. The mixture was incubated in water bath at 65° C. for 20 min and immediately immersed in cool water to quench the reaction. The reaction samples (200 μL/well) were then taken in 96-well plate to read the absorbance at 560 nm using a microplate reader (Tecan Infinite MPro). The concentration of Hyp was determined using the Hyp standard curve.

The pharmacokinetic parameters were determined using the non-compartmental analysis. The data were analyzed by independent t-test (GraphPad Prism version 10.1) and presented as mean±standard deviation (SD).

Table 6 shows the pharmacokinetics of encapsulated collagen versus control. The pharmacokinetic data showed that Hyp had significantly higher Cₘₐₓ values in encapsulated collagen administered rats (9.15±1.05 mg/mL, p=0.001) than the non-encapsulated control group (3.91±0.04 mg/mL). Further, the Tₘₐₓ and t_{1/2} values were higher in the encapsulated collagen group than the non-encapsulated control group. Interestingly, the AUC of plasma concentration of Hyp vs time in the encapsulated collagen group (130.10±3.16 mg·h/mL, p<0.0001) was significantly higher than the non-encapsulated control group (51.45±5.39 mg·h/mL). The relative bioavailability of encapsulated collagen compared to the non-encapsulated control was 2.54±0.20.

TABLE 6
Pharmacokinetic Parameters of Plasma Concentration of Hyp in
Rat Plasma (n = 3) After Ingestion of Collagen Formulations

Oral administration

| | | Unformulated |
|---|---|---|
| Parameter | FIL-collagen | collagen |
| Cₘₐₓ, mg/mL | 9.15 ± 1.05†** | 3.91 ± 0.04 |
| Tₘₐₓ, h | 0.42 ± 0.14†^{ns} | 0.33 ± 0.14 |
| AUC, h × mg/mL | 130.10 ± 3.16†*** | 51.45 ± 5.39 |
| t_{1/2}, h | 63.25 ± 20.22†^{ns} | 30.09 ± 20.18 |
| Fᵣ | 2.54 ± 0.20 | — |

Values are mean ± standard deviation.
†Independent t-test.
**p ≤ 0.001,
***p ≤ 0.0001;
ns: not significant

In summary, this study provides information on the plasma kinetics after a single oral dose of collagen formulated with the inventive liposome. The results from this comparative study demonstrate the encapsulated formulation markedly improves the oral bioavailability of collagen relative to non-encapsulated collagen supplementation in vivo.

1. A composition comprising liposomes wherein said liposomes comprise one or more types of fiber.

2. The liposome of claim 1, wherein said composition is substantially free of water.

3. The liposome of claim 1, wherein said composition is powdered.

4. The composition of claim 1, wherein said liposomes are formed from one or more types of phospholipid.

5. The composition of claim 4, wherein said one or more types of phospholipid include one or more phospholipids selected from glycerophospholipids and phosphosphingolipids.

6. The composition of claim 4, wherein said one or more types of phospholipid includes lecithin.

7. The composition of claim 1, wherein said one or more types of fiber include dietary fiber.

8. The composition of claim 7, wherein said dietary fiber includes fiber selected from insoluble dietary fiber, soluble dietary fiber, and a combination thereof.

9. The composition of claim 7, wherein said dietary fiber includes fiber selected from oat fiber, pea fiber, bean fiber, apple fiber, citrus fruit fiber, carrot fiber, barley fiber, psyllium fiber, and combinations thereof.

10. The composition of claim 1, wherein said one or more types of fiber includes one or more types of amphiphilic fiber.

11. The composition of claim 1, wherein said liposomes have a phospholipid bilayer and said one or more types of fiber are (a) interposed between phospholipid molecules in said phospholipid bilayer, (b) encapsulated within said liposome, (c) on an outside surface of said bilayer, (d) on an inside surface of said bilayer, or (e) combinations thereof.

12. The composition of claim 1, wherein said liposomes encapsulate at least one agent.

13. The composition of claim 12, wherein said at least one agent includes an agent selected from vitamins, minerals, phytomolecules, biomolecules, withanolides, steroids, small molecule drugs, and combinations thereof.

14. The composition of claim 13, wherein said vitamins include a vitamin selected from vitamin C, vitamin B, vitamin D, vitamin A, vitamin E, vitamin K, and combinations thereof.

15. The composition of claim 13, wherein said phytomolecules include phytomolecules selected from flavonoids, polyphenols, phenolic acids, isoflavones, curcumin, resveratrol, isothiocyanates, carotenoids and combinations thereof.

16. The composition of claim 12, wherein said at least one agent includes one or more botanical extracts.

17. The composition of claim 16, wherein said one or more botanical extracts include one or more botanical extracts selected from green coffee bean extract, sunflower seed extract, turmeric rhizome extract, coffee fruit pulp extract, jackfruit extract, mango extract, Aframomum melegueta extract, and combinations thereof.

18. The composition of claim 1, wherein said composition further comprises starch.

19. The composition of claim 18, wherein said starch coats said liposomes.

20. The composition of claim 18, wherein said liposomes comprise 10-40% (w/w) phospholipid, 3-15% (w/w) of said one or more types of fiber and 10-60% (w/w/) of said starch.
